(19) United States
(12) Plant Patent Application Publication
(10) Pub. No.: US 2017/0223884 P1
(43) Pub. Date: Aug. 3, 2017

(54) Citrus reticulata blanco plant named 'TAMDO NO.3'

(50) ;

(71) Applicant: THE RURAL DEVELOPMENT ADMINISTRATION, REPUBLIC OF KOREA, Chungcheongnam-Do (KR)

(72) Inventors: Jaeho Park, Seogwipo-Si (KR); Su-Hyun Yun, Jeollabuk-Do (KR); Sangwoog Koh, Jeollabuk-Do (KR); Minju Kim, Seogwipo-Si (KR); Yonghun Choi, Seogwipo-Si (KR); Donghoon Lee, Jeollabuk-Do (KR); I-Ung Yang, Seogwipo-Si (KR); Chiwon Chae, Seogwipo-Si (KR)

(21) Appl. No.: 14/998,683

(22) Filed: Feb. 2, 2016

(51) Int. Cl. A01H 5/08

(52) U.S. Cl. PLT/201; PLT/202

(57) ABSTRACT

A new and distinct Citrus Reticulate Blanco plant named ‘Tamdo No. 3’, characterized by its upright plant type and strong vigor, The plant type of Tamdo No. 3 is upright and the tree vigor is strong, fruit shape is oblate and the fruit shape index is about 143, fruit skin thickness is about 2.7 mm so it is easy skinning, the fruit flesh rate is about 82.9%, sugar of fruit juice is about 13.7° Bx, the acidity is about 0.79%, and the sugar acid ratio is about 17.34.

Latin name of the genus and species of the plant claimed: Citrus reticulata Blanco.

Variety denomination: ‘Tamdo No.3’.

### BACKGROUND OF THE INVENTION

The new and distinct cultivar is a product of a breeding program by the inventors by cross a Japanese variety ‘Kiyomi’ and an American variety ‘Sunburst’ mandarin (as the pollen parent) was carried out. The seeds obtained by the cross were sowed in the next spring. The plants started fruiting in 2010. In 2011, the first screening was carried out, and the one called Jegamna-27 was selected. In the last screening which carried out in 2012, Jegamna-27 was selected as an office upbringing new variety and named ‘Tamdo No.3’.

After Hybridization of the parents, the variety was planted by asexual propagation for three generations and there was no genetic variation, therefore its traits are stable.

The fruit characteristics of Tamdo No.3 are in the Table 1 below. The test carried out from 2010 to 2012, in Citrus Research Station.

| Survey | Fruit | | Fruit | Thickness | Seed | | | |
|---|---|---|---|---|---|---|---|---|
| date | weight | Flesh | shape | of the skin | grain | Sugar | Acidity | Sugar |
| (yyyy.mm.dd) | (g) | rate (%) | index | (mm) | number | (° Bx) | (%) | acid ratio |
| 2010. 1. 6 | 254.2 | 85.9 | 155 | 1.7 | 0.0 | 10.8 | 0.82 | 13.19 |
| 2010. 3.22 | 173.9 | 77.9 | | 3.0 | 2.4 | 11.8 | 1.22 | 9.66 |
| 2011. 1. 5 | 189.8 | 85.1 | 157 | 1.5 | 0.6 | 12.9 | 1.28 | 10.13 |
| 2011. 1.20 | 213.8 | 82.3 | 155 | 2.1 | 0.0 | 12.7 | 1.28 | 10.46 |
| 2011. 2. 7 | 200.8 | 81.9 | 149 | 2.7 | 0.0 | 14.0 | 1.09 | 13.02 |
| 2011. 2.21 | 171.6 | 82.7 | 152 | 2.3 | 0.0 | 14.4 | 1.31 | 11.20 |
| 2011. 3. 8 | 232.6 | 78.5 | 152 | 2.8 | 0.0 | 14.7 | 1.06 | 13.85 |
| 2011. 3.29 | 262.2 | 81.4 | 158 | 2.4 | 0.0 | 13.5 | 0.85 | 16.05 |
| 2012. 2. 6 | 290.8 | 84.6 | 149 | 2.3 | 0.0 | 12.9 | 0.88 | 14.69 |
| 2012. 2.20 | 280.0 | 82.9 | 143 | 2.7 | 0.0 | 13.7 | 0.79 | 17.34 |
| 2012. 3.13 | 284.6 | 82.6 | 141 | 2.2 | 0.0 | 13.8 | 0.82 | 16.77 |

Fruit shape index = The transverse diameter ÷ The longitudinal diameter × 100

### SUMMARY OF THE INVENTION

The cultivar ‘Tamdo No. 3’ has not been observed under every possible environmental conditions. Therefore, there may be variations in the phenotype due to varying environmental conditions such as temperature, day length and light intensity. However, the genotype will not vary.

The following trait have been repeatedly observed and are determined to be in the characteristics of the new cultivar of Citrus Reticulata Blanco. These attributes in combination distinguish ‘Tamdo No. 3’ as unique from and all other varieties of Citrus reticulate Blando know to inventors.

1. The plant type of Tamdo No.3 is upright and the tree vigor is strong.

2. The fruit color is orange.

3. Germination stage of Tamdo No.3 is in the middle of April, and the blooming stage is in late May.

4. Fruits start coloring in late October and finish coloring in early December.

5. The mature stage is in the middle of February.

6. Fruit shape is oblate and the fruit shape index is about 143.

7. Fruit skin thickness is about 2.7 mm so it is easy skinning.

8. The fruit flesh rate is about 82.9%.

9. Sugar of fruit juice is about 13.7° Bx, the acidity is about 0.79%, and the sugar acid ratio is about 17.34.

Plants of the new cultivar ‘Tamdo No. 3’ are similar to plants of the seed parent ‘Kiyomi’ in most horticultural characteristics, however, the plants of the new cultivar ‘Tamdo No. 3’ produce tree vigor that are stronger than the parent. Compared to the parent, ‘Tamdo No. 3’ produces fruit with skin thickness of about 2.7 mm. The fruit surface of the new cultivar is smooth, whereas the ‘Kiyomi’ cultivar has a rough fruit surface.

The new cultivar differs from the closest comparable plants and characteristics of the new cultivar as shown in the tables below:

TABLE 2

| Denomination | Characteristic in | The expression of the characteristic | |
|---|---|---|---|
| of similar | which the similar | | Tamdo No. 3 |
| variety | variety is different | Kiyomi Variety | Variety |
| Kiyomi | plant: type | medium | upright |
| | Tree vigor | medium | strong |
| | Fruit: shape index | 119(round) | 143(oblate) |
| | Fruit: flesh rate | 74.6% | 82.9% |
| | Fruit: skin thickness | 4.9 mm | 2.7 mm |
| | Fruit: skin surface | rough | smooth |
| | Fruit juice: Sugar | 9.7 | 13.7 |
| | (° Bx) | | |
| | Fruit juice: Acidity | 1.17 | 0.79 |
| | (%) | | |
| | Fruit: sugar acid | 8.29 | 17.34 |
| | ratio | | |
| | Fruit: color | yellowish | medium orange |
| | | orange | |
| | Mature stage | early (in the | very early (in |
| | | middle | the middle of |
| | | of March) | February) |

TABLE 3

| | | | | Thickness | | | | | Degree | |
|---|---|---|---|---|---|---|---|---|---|---|
| | Fruit | | Fruit | of the | Seed | | | Sugar | of | Survey |
| | weight | Flesh | shape | skin | grain | Sugar | Acidity | acid | color | date |
| Variety | (g) | rate (%) | index | (mm) | number | (° Bx) | (%) | ratio | (a*) | (yyyy.mm.dd) |
| Tamdo | 213.8 | 82.3 | 155 | 2.1 | 0.0 | 12.7 | 1.28 | 10.46 | 30.8 | 2011. 1.20 |
| No.3 | | | | | | | | | | |
| Kiyomi | 214.0 | 74.4 | 107 | 4.1 | 0.0 | 10.1 | 1.75 | 5.99 | 18.7 | 2011. 1.20 |
| T-Test | NS | * | * | * | | * | NS | * | * | |
| (0.05) | | | | | | | | | | |
| Tamdo | 232.6 | 78.5 | 152 | 2.8 | 0.0 | 14.7 | 1.06 | 13.85 | 33.6 | 2011. 3. 8 |
| No.3 | | | | | | | | | | |
| Kiyomi | 209.9 | 70.5 | 118 | 4.8 | 0.0 | 10.3 | 1.55 | 6.68 | 22.6 | 2011. 3. 9 |
| T-Test | NS | * | * | * | | * | * | * | * | |
| (0.05) | | | | | | | | | | |
| Tamdo | 280.0 | 82.9 | 143 | 2.7 | 0.0 | 13.7 | 0.79 | 17.34 | 31.1 | 2012. 2.20 |
| No.3 | | | | | | | | | | |
| Kiyomi | 318.7 | 74.6 | 119 | 4.9 | 0.0 | 9.7 | 1.17 | 8.29 | 18.7 | 2012. 2.20 |
| T-Test | NS | * | * | * | | * | * | * | * | |
| (0.05) | | | | | | | | | | |

TABLE 4

| | | | | | | | | | | | Application | | Similar | |
|---|---|---|---|---|---|---|---|---|---|---|---|---|---|---|
| | | | | | | | | | | | variety: | | variety: | |
| | | Forms of Expression | | | | | | | | | Tamdo No.3 | | Kiyomi | |
| NO | Characteristics | 1 | 2 | 3 | 4 | 5 | 6 | 7 | 8 | 9 | No | Data | No | Data |
| 1 | Chromosome | | 2 | 3 | 4 | | | | | | 2 | | 2 | |
| | multiple | | | | | | | | | | | | | |
| 2 | Plant: Type | upright | | | | medium | | | | pendulous | 1 | | 5 | |
| 3 | Plant: density | no or | | | | medium | | | | densely | 1 | | 1 | |
| | of spines | sparse | | | | | | | | | | | | |
| 4 | leaf: color | light | | | | green | | | | dark | 9 | | 9 | |
| | | green | | | | | | | | green | | | | |
| 5 | leaf: length | very | | short | | medium | | long | | very | 5 | 10.3 cm | 7 | 12.7 cm |
| | | short | | | | | | | | long | | | | |
| 6 | leaf: width | very | | narrow | | medium | | broad | | very | 5 | 4.8 cm | 7 | 5.8 cm |
| | | narrow | | | | | | | | broad | | | | |
| 7 | leaf: | small | | | | medium | | | | large | 5 | 2.2 | 5 | 2.2 |
| | length/width | | | | | | | | | | | | | |
| | ratio | | | | | | | | | | | | | |
| 8 | Leaf petiole: | very | | short | | medium | | long | | very | 3 | 14.9 mm | 5 | 21.7 mm |
| | length | short | | | | | | | | long | | | | |
| 9 | Flower: calyx | small | | | | medium | | | | large | 5 | | 5 | |
| | diameter | | | | | | | | | | | | | |
| 10 | Flower: petal | very | | short | | medium | | long | | very | 5 | 18.2 mm | 5 | 16.1 mm |
| | length | short | | | | | | | | long | | | | |
| 11 | Flower: petal | very | | narrow | | medium | | broad | | very | 5 | 8.9 mm | 5 | 7.7 mm |
| | width | narrow | | | | | | | | broad | | | | |
| 12 | Flower: petal | small | | | | medium | | | | large | 5 | 2.0 | 5 | 2.1 |
| | length/width | | | | | | | | | | | | | |
| | ratio | | | | | | | | | | | | | |
| 13 | Flower: stamen | very | | short | | medium | | long | | very | 5 | | 5 | |
| | length | short | | | | | | | | long | | | | |
| 14 | Flower: anther | white | | | | light | | | | medium | 1 | | 1 | |
| | color | | | | | yellow | | | | yellow | | | | |
| 15 | Flower: activity | absent | | | | | | | | present | 1 | | 1 | |
| | pollen | | | | | | | | | | | | | |
| 16 | Fruit: length | very | | short | | medium | | long | | very | 5 | 59.7 mm | 7 | 73.5 mm |
| | | short | | | | | | | | long | | | | |
| 17 | Fruit: width | very | | narrow | | medium | | large | | very | 7 | 89.4 mm | 7 | 84.3 mm |
| | | narrow | | | | | | | | large | | | | |
| 18 | Fruit: | small | | | | medium | | | | large | 5 | 0.67 | 9 | 0.87 |
| | length/width | | | | | | | | | | | | | |
| | ratio | | | | | | | | | | | | | |
| 19 | Fruit: neck | absent | | | | | | | | present | 9 | | 1 | |
| 20 | Fruit: areola | absent | | | | | | | | present | 1 | | 1 | |
| 21 | Fruit: surface | green | yellowish | light | medium | yellowish | medium | dark | red with | red | 6 | 31.8(a) | 5 | 20.0(a) |
| | color | | green | yellow | yellow | orange | orange | orange | orange | | | | | |
| 22 | Fruit: surface | smooth | | | | | | | | rough | 1 | | 9 | |
| | roughness | | | | | | | | | | | | | |
| 23 | Fruit: fresh | with | light | light | medium | light | medium | dark | red | purple | 6 | | 4 | |
| | color | white | green | yellow | yellow | orange | orange | orang | | | | | | |
| 24 | Fruit: skin | thin | | | | medium | | | | thick | 5 | 2.7 mm | 9 | 4.9 mm |
| | thickness | | | | | | | | | | | | | |
| 25 | Fruit: skin | weak | | | | medium | | | | strong | 5 | | 9 | |
| | strength | | | | | | | | | | | | | |
| 26 | Fruit juice: total | low | | | | medium | | | | high | 9 | 13.7° Bx | 5 | 9.7° Bx |
| | soluble solids | | | | | | | | | | | | | |
| 27 | Fruit juice: acid | low | | | | medium | | | | high | 1 | | 5 | |
| 28 | Mature stage | very | | early | | medium | | late | | very | 1 | | 3 | |
| | | early | | | | | | | | late | | | | |
| 29 | Parthenocarpy | absent | | | | | | | | present | 9 | | 9 | |
| 30 | Self- | absent | | | | | | | | present | 9 | | 9 | |
| | incompatibility | | | | | | | | | | | | | |
| 31 | Tree vigor | weak | | | | medium | | | | strong | 9 | | 5 | |

### BRIEF DESCRIPTION OF THE DRAWINGS

The accompanying colored photographs illustrate the overall appearance and distinct characteristics of the new Citrus reticulate Blanco ‘Tamdo No. 3’ showing colors as true as is reasonably possible with colored reproduction of this type. These photos were taken in 2012, in Korea. Colors in the photographs may differ slightly from the color values cited in the detained botanical description which accurately describes the color of the ‘Tamdo No. 3’.

FIG. 1 provides multiple views of typical fruits harvested from the cultivar ‘Tamdo No. 3’. Top, bottom and side views of the whole, unpeeled fruit are shown as well as two cross-sectional views of the inside of the fruit.

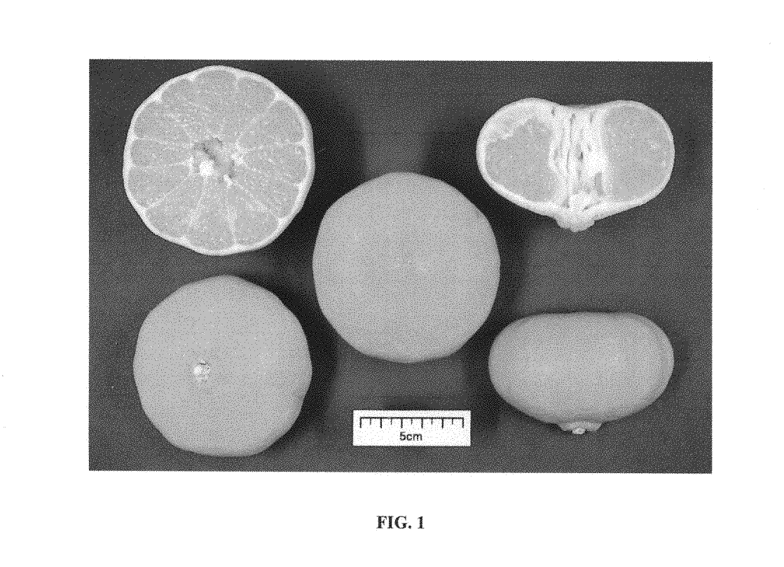

FIG. 2 illustrates in full color typical foliage and fruits of ‘Tamdo No. 3’ on the tree.

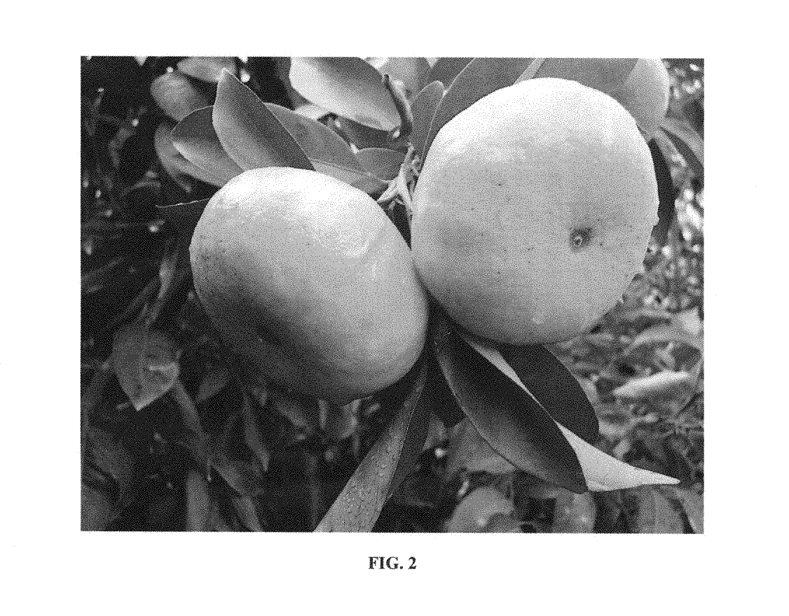

### DETAILED BOTANICAL DESCRIPTION

The following is a detailed description of the new cultivar ‘Tamdo No. 3’ plants as grown in Korea. The descriptions were taken from 2010 to 2012.
Botanical classification: Citrus reticulate Blanco ‘Tamdo No. 3’.
Propagation: ‘Tamdo No. 3’ is typically grafted onto rootstock of ‘Jegamna-27’.
Plant:
   Age of the plant described.—Approximately 3 years.
   Chromosome multiples.—2.
   Plant type.—Upright.
   Plant tree vigor.—Strong.
   Plant density of spines.—No or sparse.
Foliage:
   Leaf color.—Dark green.
   Leaf length.—About10.3 cm.
   Leaf width.—About 4.8 cm.
   Leaf length/width ratio.—About 2.2.
   Leaf petiole length.—About 14.9 mm.
Flower:
   Flower calyx diameter.—Medium.
   Flower petal length.—About 18.2 mm.
   Flower petal width.—About 8.9 mm.
   Flower petal length/width ratio.—About 2.0.
   Flower stamen length.—Medium.
   Flower anther color.—White.
   Flower activity pollen.—Absent.
Fruit:
   Fruit length.—About 59.7 mm.
   Fruit width.—About 89.4 mm.
   Fruit length/width ratio.—About 0.67.
   Fruit neck.—Present.
   Fruit areola.—Absent.
   Fruit surface color.—Medium orange.
   Fruit surface roughness.—Smooth.
   Fruit fresh color.—Medium orange.
   Fruit skin thickness.—About 2.7 mm.
   Fruit skin strength.—Medium.
   Fruit flesh rate.—About 82.9%.
   Fruit sugar acid ratio.—About 17.34.
   Fruit juice.—Total soluble solids: about 13.7° Bx.
   Fruit juice acidity.—About 0.79%.
   Mature stage.—Very early.
   Parthenocarpy.—Present.
   Self incompatibility.—Present.
   Tree vigor.—Strong.
   Other fruit features.—For the very early mature stage, Tamdo No.3 should be protected cultured. Pruning on Tamdo No.3 is as the same as other variety which belongs to Citrus reticulata Blanco.

What is claimed is:

1. A new and distinct cultivar of Citrus reticulate Blanco plant named ‘Tamdo No. 3’ as herein illustrated and described.
